SEQUENCE LISTING (33 sequences)

SEQ ID NO: 1 - DNA, 42 bp, Arabidopsis thaliana
tctagaaggaggattacaaaatggcggcaggactctcaaccg

SEQ ID NO: 2 - DNA, 42 bp, Arabidopsis thaliana
tctagaaggaggattacaaaatgatctcaatgttggaaatgt

SEQ ID NO: 3 - DNA, 30 bp, Arabidopsis thaliana
gtcaatcgtctctagagtgcttgctacacg

SEQ ID NO: 4 - DNA, 35 bp, Arabidopsis thaliana
gaatcgcctacatgtttgtccacgatggtctcgtg

SEQ ID NO: 5 - DNA, 35 bp, Arabidopsis thaliana
cacgagaccatcgtggacaaacatgtaggcgattc

SEQ ID NO: 6 - DNA, 28 bp, Arabidopsis thaliana
gaagaacggagacagaattctccgattg

SEQ ID NO: 7 - DNA, 49 bp, Arabidopsis thaliana
gccgactctagaaggaggattacaaaatggaagaacggagacagaattc

SEQ ID NO: 8 - DNA, 29 bp, Arabidopsis thaliana
tctttctcctcctcctctaccgatttccg

SEQ ID NO: 9 - DNA, 47 bp, Arabidopsis thaliana
gccgactctagaaggaggattacaaaatgtctttctcctcctcctct

SEQ ID NO: 10 - DNA, 47 bp, Pantoea ananatis
gccgcctctagaaggaggattacaaaatgttgtggatttggaatgcc

SEQ ID NO: 11 - DNA, 39 bp, Pantoea ananatis
ctggtcccatggttacttcccggatgcgggctcatcctc

SEQ ID NO: 12 - DNA, 18 bp, Escherichia coli
agtcagtgagcgaggaag

SEQ ID NO: 13 - DNA, 18 bp, Escherichia coli
cggcatcagagcagattg

SEQ ID NO: 14 - DNA, 40 bp, Arabidopsis thaliana
gagccgaagcttaacaaaatggcggcaggactctcaaccg

SEQ ID NO: 15 - DNA, 35 bp, Arabidopsis thaliana
ggatcactcgagtcaagaactcgaactcgacccgg

SEQ ID NO: 16 - DNA, 39 bp, Pantoea ananatis
ggcgccaagcttaacaaaatgttgtggatttggaatgcc

SEQ ID NO: 17 - DNA, 30 bp, Pantoea ananatis
atatatctcgagttacttcccggatgcggg

SEQ ID NO: 18 - DNA, 18 bp, Saccharomyces cerevisiae
cggatcggactactagca

SEQ ID NO: 19 - DNA, 18 bp, Saccharomyces cerevisiae
aatcgagaccgaggagag

SEQ ID NO: 20 - DNA, 32 bp, Pantoea ananatis
atgtcagcatgcccacacccgccgcgcttaat

SEQ ID NO: 21 - DNA, 32 bp, Pantoea ananatis
gtagacgcatgccgcgcgttggccgattcatt

SEQ ID NO: 22 - DNA, 31 bp, Pantoea ananatis
gaattcaggaggattaacaaaatgcaaccgc

SEQ ID NO: 23 - DNA, 25 bp, Pantoea ananatis
ccgcagcggtcgaccatgggaattc

SEQ ID NO: 24 - DNA, 20 bp, Pantoea ananatis
gacagtttggcccgcacttg

SEQ ID NO: 25 - DNA, 20 bp, Pantoea ananatis
caagtgcgggccaaactgtc

SEQ ID NO: 26 - DNA, 20 bp, Pantoea ananatis
ccgcctgagtgcacttgatg

SEQ ID NO: 27 - DNA, 20 bp, Pantoea ananatis
catcaagtgcactcaggcgg

SEQ ID NO: 28 - DNA, 36 bp, Pantoea agglomerans
gtcgacaacaaagatggaaaaaaccgttgtgattgg

SEQ ID NO: 29 - DNA, 28 bp, Pantoea agglomerans
tggccatggtcgacgtggcgtggtcaag

SEQ ID NO: 30 - DNA, 20 bp, Pantoea agglomerans
cggcgctttctggcttactc

SEQ ID NO: 31 - DNA, 20 bp, Pantoea agglomerans
gagtaagccagaaagcgccg

SEQ ID NO: 32 - DNA, 20 bp, Pantoea agglomerans
gctgcgccagcttctacgtg

SEQ ID NO: 33 - DNA, 20 bp, Pantoea agglomerans
cacgtagaagctggcgcagc

(19) United States
(12) Patent Application Publication
(10) Pub. No.: US 2008/0124755 A1
(43) Pub. Date: May 29, 2008

(54) Biosynthesis of beta-cryptoxanthin in microbial hosts using an Arabidopsis thaliana beta-carotene hydroxylase gene

(76) Inventors: Michael Tai-Man Louie, Iowa City, IA (US); Emily-Jean Fuerst, Ames, IA (US)

Correspondence Address: DAVIS, BROWN, KOEHN, SHORS & ROBERTS, P.C.;THE FINANCIAL CENTER, 666 WALNUT STREET, SUITE 2500, DES MOINES, IA, 50309-3993, US

(21) Appl. No.: 11/546,702

(22) Filed: Oct. 12, 2006

(51) Int. Cl. C12P 23/00

(52) U.S. Cl. 435/67

(57) ABSTRACT

A method is disclosed for preparing β-cryptoxanthin from a microorganism transformed with the β-carotene hydroxylase gene from Arabidopsis thaliana by culturing the transformant in a medium and recovering β-cryptoxanthin from the resultant culture. Both bacteria and yeasts were successfully transformed. Surprisingly, β-cryptoxanthin is the dominant carotenoid produced by the transformant and was not significantly converted into zeaxanthin.

### BACKGROUND OF THE INVENTION

The invention relates generally to β-cryptoxanthin and, more specifically, to β-cryptoxanthin produced by a microbial host modified to include a β-carotene hydroxylase gene from Arabidopsis thaliana.

Carotenoids are a diverse group of natural pigments produced by plants, bacteria, and fungi. These pigments play an important protective function in quenching harmful singlet oxygen molecules, reactive oxygen species, and free radicals that are metabolic by-products in cells that cause oxidative damage [Krinsky N. I. (1994) The biological properties of carotenoids. Pure Appl. Chem. 66:1003-1010]. Hydroxylated carotenoids, such as lutein, zeaxanthin, and β-cryptoxanthin, are members of the xanthophylls class of carotenoids [Bhosale P and Bernstein P S (2005) Microbial xanthophylls. Appl. Microbiol. Biotechnol. 68:445-455]. β-cryptoxanthin differs from its xanthophyll counterparts by having only one rather than two hydroxyl groups. β-cryptoxanthin has strong antioxidant activity and mild provitamin A activity, which is a potent anti-cancer agent that inhibits cell proliferation and stimulates cell differentiation [Melendez-Martinez A. J., Vicario I. M., and Heredia F. J. (2004) Nutritional importance of carotenoid pigments. Arch. Latinoam. Nutr. 54:149-154]. β-Cryptoxanthin is the fourth most abundant carotenoid found in human serum [Khachik F., Spangler C. J., and Smith J. C. (1997) Identifications, quantification, and relative concentrations of carotenoids and their metabolites in human milk and serum. Anal. Chem. 69:1873-1881] and is present in breast milk [Khachik], cervical [Peng Y. M., Peng Y. S., Childers J. M., Hatch K. D., Roe D. J., Lin Y., and Lin P. (1998) Concentrations of carotenoids, tocopherols, and retinal in paired plasma and cervical tissue of patients with cervical cancer, precancer, and noncancerous diseases. Cancer Epidemiol. Biomarkers Prev. 7:347-350], breast [Yeum K. J., Ahn S. H., Rupp de Paiva S. A., Lee-Kim Y. C., Krinsky N. I., and Russell R. M. (1998) Correlation between carotenoid concentrations in serum and normal breast adipose tissue of women with benign breast tumor or breast cancer. J. Nutr. 128:1920-1926], prostrate [Clinton S. K., Emenhiser C., Schwartz S. J., Bostwick D. G., Williams A. W., Moore B. J., and Erdman J. W. Jr. (1996) Cis-trans lycopene isomers, carotenoids, and retinal in the human prostate. Cancer Epidemiol. Biomarkers Prev. 5:823-833], skin [Wingerath T., Sies H. and Stahl W. (1998) Xanthophyll esters in human skin. Arch. Biochem. Biophys. 355:271-274], and ocular tissues [Bernstein P. S., Khachik F., Carvalho L. S., Muir G. J., Zhao D. Y., and Katz N. B. (2001) Identification and quantitation of carotenoids and their metabolites in the tissues of the human eye. Exp. Eye Res. 72:215-223]. β-Cryptoxanthin is supported by many scientific studies to be beneficial to human health. A recent cohort study showed that high prediagnostic levels of β-cryptoxanthin in the serum are associated with reduced risk of lung cancer [Yuan J. M., Ross R. K., Chu X., Gao Y. T., and Yu M. C. (2001) Prediagnostic levels of serum β-cryptoxanthin and retinal predict smoking-related lung cancer risk in Shanghai, China. Cancer Epidemiol. Biomark Prev. 10:767-773; Yuan J. M., Stram D. O., Arakawa K., Lee H. P., and Yu M. C. (2003) Dietary cryptoxanthin and reduced risk of lung cancer: the Singapore Chinese health study. Cancer Epidemiol Biomarkers Prev. 12:890-898]. Epidemiological and experimental studies have shown β-cryptoxanthin to aid in the prevention of prostate cancer [Binns C. W., Jian L. J. L., and Lee A. H. (2004) The relationship between dietary carotenoids and prostate cancer risk in Southeast Chinese men. Asia Pac. J. Clin. Nutr. 13:S117], colon cancer [Sumida T. (2002) Heath claims of β-crypoxanthin-rich juice from Satsuma mandarin. Shokuhin Kogyo 45:18-26], and rheumatoid arthritis [Pattison D. J., Harrison R. A., and Symmons D. P. M. (2004) The role of diet in susceptibility to rheumatoid arthritis: a systematic review. J. Rheumatol. 31:1310-1319]. In animal studies, oral administration of β-cryptoxanthin was associated with a preventative effect of bone loss with increasing age and osteoporosis [Uchiyama S., Sumida T., and Yamaguchi M. (2004) Oral administration of β-cryptoxanthin induces anabolic effects on bone components in the femoral tissues of rats in vivo. Biol. Pharm. Bull. 27:232-235; Uchiyama S. and Yamaguchi M. (2005a) Oral administration of β-cryptoxanthin prevents bone loss in streptozotocin-diabetic rats in vivo. Biol. Pharm. Bull. 28:1766-1769]. It was shown that β-cryptoxanthin stimulates osteoblastic bone formation and inhibits osteoclastic bone resorption in vitro [Uchiyama S. and Yamaguchi M. (2004) Inhibitory effect of beta-cryptoxanthin on osteoclast-like cell formation in mouse marrow cultures. Biochem. Pharmacol. 67:1297-1305; Uchiyama S. and Yamaguchi M. (2005b) β-cryptoxanthin stimulates cell differentiation and mineralization in osetoblastic MC3T3-E1 cells J. Cell. Biochem. 95:1224-1234; Uchiyama S. and Yamaguchi M. (2005c) δ-cryptoxanthin stimulates cell proliferation and transcriptional activity in osteoblastic MC3T3-E1 cells. Int. J. Mol. Med. 15:675-681; Yamaguchi M. and Uchiyama S. (2004) β-cryptoxanthin stimulates bone formation and inhibits bone resorption in tissue culture in vitro. Mol. Cell. Biochem. 258:137-144].

Dietary sources of β-cryptoxanthin include fruits such as oranges, tangerines, papayas, and mangos [Chug-Ahuja J. K., Holden J. M., Forman M. R., Mangels A. R., Beecher G. R., and Lanza E. (1993) The development and application of a carotenoid database for fruits, vegetables, and selected multicomponent foods. J. Am. Diet. Assoc. 93:318-323; Mangels A. R., Holden J. M., Beecher G. R., Forman M. R., and Lanza E. (1993) Carotenoids content of fruits and vegetables: an evaluation of analytic data. J. Am. Diet. Assoc. 93:284-296]; however, β-cryptoxanthin is present in trace amounts in these natural sources, with the highest reported concentration of approximately 0.005% (w/w) [Kim I. J., Ko K. C., Ko C. S., and Chung W. I. (2001) Isolation and characterization of cDNAs encoding β-carotene hydroxylase in Citrus. Plant Sci. 161:1005-1010]. This low available concentration of natural β-cryptoxanthin prevents commercialization of this molecule by traditional solvent-based extraction procedures. The lack of a microorganism that naturally produces β-cryptoxanthin as the final product also prohibits using fermentation technology for commercial production of β-cryptoxanthin. An alternative method to produce commercially viable concentrations of β-cryptoxanthin is needed and genetically engineering of a recombinant microorganism to produce high levels of β-cryptoxanthin would be a solution.

### SUMMARY OF THE INVENTION

The present invention consists of β-cryptoxanthin produced in a non-carotenogenic Escherichia coli by an engineered metabolic pathway utilizing a β-carotene hydroxylase from Arabidopsis thaliana (GenBank accession no. for the protein: NP_{—}11149300). When a full-length β-carotene hydroxylase gene (crtZ) from A. thaliana was expressed in a β-carotene-producing E. coli platform, surprisingly β-cryptoxanthin was found to accumulate inside the cells without being further converted to zeaxanthin. The present invention also consists of β-cryptoxanthin produced in a non-carotenogenic Saccharomyces cerevisiae by an engineered metabolic pathway utilizing β-carotene hydroxylase from Arabidopsis thaliana.

### BRIEF DESCRIPTION OF THE FIGURES

FIG. 1 is a schematic diagram of the β-cryptoxanthin biosynthetic pathway.

FIGS. 2A-2D are HPLC charts of carotenoid extracts from E. coli JM109(pACmod-EBI₁₄Y) plus: (A) pUCmod-At-crtZ, (B) pUCmod-At-crtΔZ, (C) pUCmod-At-crtZ′4, and (D) no plasmid; cells were harvested after 24 hours of growth and extracted for carotenoids.

FIGS. 3A and 3B are growth curves and HPLC peak areas of β-carotene, β-cryptoxanthin, and zeaxanthin normalized against dry cell pellet weight from (A) JM109(pACmod-EBI₁₄Y/pUCmod-At-crtZ) and (B) JM109(pACmod-EBI₁₄Y/pUCmod-At-crtΔZ) grown under batch mode fermentation.

FIG. 4 is an alignment of the CrtZ protein encoded by Arabidopsis thaliana and Pantoea ananatis using ClustalW; residues in Exon 1 of the Arabidopsis protein are highlighted in black; the arrow indicates the predicted cleavage site of the chloroplast transit peptide.

FIGS. 5A-5E are HPLC analysis charts of carotenoid extracts from E. coli JM109(pACmod-EBI₁₄Y) with: (A) pUCmod, (B) pUCmod-Pa-crtZ, (C) pUCmod-At-crtZ, (D) pUCmod-At-crtZ2, (E) pUCmod-At-crtZ3.

FIGS. 6A-6E are HPLC analyses of carotenoid extracts from S. cerevisiae INVSc-1(pARC1520) with (A) pARC145G, (B) pARC145G-At-crtZ, and (C) pARC145G-Pa-crtZ, plus YPH449(pARC1520) with (D) pARC145G-At-crtZ, and (E) pARC145G-Pa-crtZ; cells were harvested after 96 hours of growth, lyophilized, and extracted for carotenoids.

FIGS. 7A-7D are HPLC analysis of carotenoid extracts from S. cerevisiae YPH499 with: (A) pARC145G-At-crtZ, p423GPD-crtY, and pESC-URA-crtI; (B) pARC145G-Pa-crtZ, p423GPD-crtY, and pESC-URA-crtI; (C) pARC145G-At-crtZ, pARC1520, and p423GPD-crtY; and (D) pARC145G-At-crtZ, pARC1520, and p423GPD-crtY.

### DETAILED DESCRIPTION OF PREFERRED EMBODIMENTS

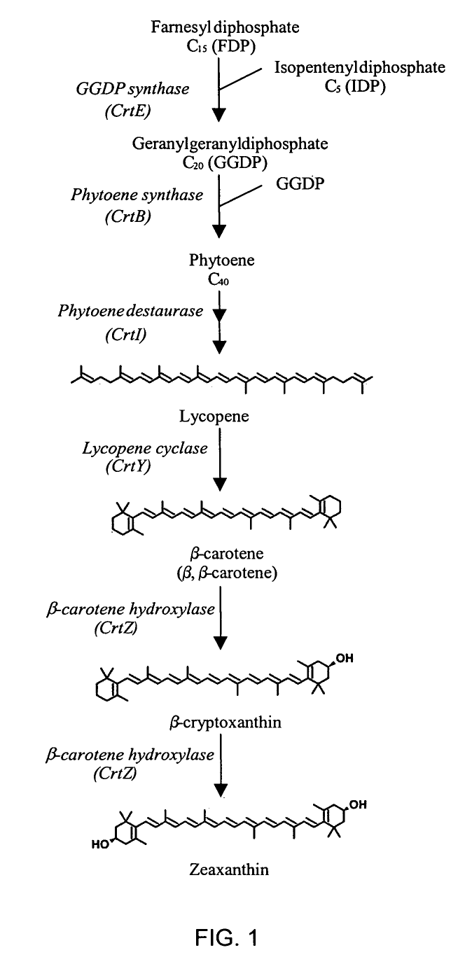

Embodiments of the present invention include the engineering of a carotenoid biosynthetic pathway for producing β-cryptoxanthin in Escherichia coli and in Saccharomyces cerevisiae. The use of E. coli and S. cerevisiae, both non-carotenogenic microorganisms, to produce β-cryptoxanthin required extending the native terpenoid pathway with genes coding for geranylgeranlydiphosphate synthase (CrtE), phytoene synthase (CrtB), and phytoene desaturase (CrtI) to produce lycopene from farnesyl diphosphate (FIG. 1). Additionally, genes coding for lycopene cyclase (CrtY) and β-carotene hydroxylase (CrtZ) are needed to produce β-cryptoxanthin from lycopene (FIG. 1). Normally, a β-carotene hydroxylase will sequentially hydroxylate both β-ionone rings of β-carotene, producing zeaxanthin. β-Cryptoxanthin is an intermediate of this reaction and thus little to no β-cryptoxanthin accumulates in vivo. However, Sun et al. [Sun Z., Gantt E., and Cunningham F. X. (1996) Cloning and functional analysis of the β-carotene hydroxylase of Arabidopsis thaliana. J. Biolo. Chem. 271:24349-24352] demonstrated that expression of a truncated Arabidopsis thaliana β-carotene hydroxylase gene (crtΔZ), in which the nucleic acid sequence coding for the first 129 amino acids was deleted, in a β-carotene-producing E. coli led to accumulation of β-cryptoxanthin inside the cells. Interestingly, when we attempted to repeat Sun et al. experiment by cloning and expressing the same truncated β-carotene hydroxylase gene in a β-carotene-producing E. coli, very little β-cryptoxanthin was produced. Instead, zeaxanthin was found to be the major carotenoid accumulated inside the cells. In contrast, data disclosed herein demonstrate that when a full-length A. thaliana β-carotene hydroxylase (crtZ) gene was expressed in the same β-carotene-producing E. coli, β-cryptoxanthin was produced as the major carotenoid inside the cells. Similar results were obtained when the full-length A. thaliana β-carotene hydroxylase (crtZ) gene was expressed in a S. cerevisiae engineered to produce β-carotene.

### DEFINITIONS

As used in this specification, the following terms have the ascribed meanings:

Anneal means the biochemical process of hybridizing or binding two segments of complementary nucleic acid at an optimal temperature.

Clone means a group of identical cells derived from a single ancestor.

Digest means restriction digest, that is, the process of cutting DNA molecules with special enzymes called restriction endonucleases.

DNA means deoxyribonucleic acid means material inside the nucleus of cells that carries genetic information.

Electroporation means a process using high-voltage current to make cell membranes permeable to allow the introduction of new DNA.

Expression means the process by which a gene's information is converted into the structures and functions of a cell; process by which a gene's information is converted into functional protein.

Extracts of cell (subsequently analyzed for carotenoids), or cell extracts means the removal or separation of product in solvent from cellular biomass.

Fuse or fused means to be joined together into a whole.

Fusion gene means a gene resulting from the joining of genes.

In-frame refers to a gene or DNA sequence that is correctly oriented with native DNA inside the open reading frame (ORF) for expression.

Ligation means a process by which two strands of DNA are joined.

Non-carotenogenic means not producing carotenoids.

PCR or polymerase chain reaction means a technique for rapidly synthesizing many copies of a specific segment of DNA.

Plasmid means an extrachromosomal, circular DNA capable of replicating that can be used as a cloning vector.

Template means a macromolecular pattern for the synthesis of another molecule, a single DNA strand that serves as a pattern for building a new second strand.

Transformed or transformant means a cell that has been modified by the application of DNA into the cell.

Vector means a self-replicating DNA molecule that transfers a DNA segment between host cells.

### EXAMPLE 1

### β-cryptoxanthin Produced in a Non-Carotenogenic Escherichia coli by an Engineered Metabolic Pathway Utilizing β-carotene Hydroxylase from Arabidopsis thaliana

Materials and Methods

Materials. All reagents were of the highest purity available and were purchased from Sigma (St. Louis, Mo.), Aldrich (Milwaukee, Wis.), and Fisher Scientific (Pittsburgh, Pa.) unless otherwise noted. PCR primers were purchased from Integrated DNA Technologies (Coralville, Iowa). Pfu DNA polymerase (Stratagene, La Jolla, Calif.) and Taq DNA polymerase (Fisher Scientific, Pittsburgh, Pa.) were used in PCR reactions. Restriction endonucleases were purchased from Invitrogen (Carlsbad, Calif.), New England Biolabs (Beverly, Mass.), and Fermentas (Hanover, Mass.). Fast-Link™ DNA ligation kit was purchased from Epicentre (Madison, Wis.).

Bacterial strains, plasmids, and media. Bacterial strains and plasmids used in this study are listed in Table 1. Escherichia coli XL1-Blue (Stratagene) and E. coli JM109 were grown in 2×YT broth (per liter: Bacto tryptone, 16 g; Bacto yeast extract, 10 g; NaCl, 5 g) or LB broth (per liter: Bacto tryptone, 10 g; Bacto yeast extract, 5 g; NaCl, 10 g). Ampicillin (100 μg/mL), carbenicillin (100 μg/mL), and chloramphenicol (50 μg/mL) were added to the media for selection of various plasmid transformants.

TABLE 1
Bacteria, DNA, and plasmids used in this Example.

| | | |
|---|---|---|
| Bacteria, DNA, or | | |
| Plasmid | Genotype and description | Source |

Bacteria

| | | |
|---|---|---|
| Escherichia coli | Host for regular cloning; | Stratagene |
| XL1-Blue | recA1 endA1 gyrA96 thi-1 hsdR17 supE44 | |
| | relA1 lac [F′ proAB lacl^{q}ZΔM15 Tn10 | |
| | (Tet^{r})] | |
| Escherichia coli | Host for expression of carotenoid | Kindly provided by |
| JM109 | biosynthetic genes; | Russell Jurenka, |
| | e14⁻(McrA⁻) recA1 endA1 gyrA96 thi-1 | Iowa State |
| | hsdR17 (rK⁻ mK⁺) supE44 relA1 Δ(lac- | University |
| | proAB) [F′ traD36 proAB lacIqZΔM15] | |

DNA

| | | |
|---|---|---|
| Arabidopsis thaliana | cDNA library of Arabidopsis thaliana | Invitrogen |
| Pantoea ananatis | Genomic DNA of Pantoea ananatis | American Type |
| | (ATCC no. 19321D) | Culture Collection |

Plasmids

| | | |
|---|---|---|
| pCR2.1-TOPO | Vector for direct TA-cloning of PCR | Invitrogen |
| | product; kanamycin and ampicillin | |
| | resistant | |
| pACmod | Low copy vector for constitutive | Schmidt-Dannert* |
| | expression of carotenoid biosynthetic | |
| | genes; chloramphenicol resistant | |
| pUCmod | Vector for constitutive expression of | Schmidt-Dannert* |
| | carotenoid biosynthetic genes; ampicillin | |
| | and carbenicillin resistant | |
| pUCmod-crtY | Vector containing P. ananatis crtY (1149 bp) | Schmidt-Dannert* |
| | in pUCmod | |
| pACmod-EBI₁₄ | Vector containing crtE, crtB, and crtI₁₄ | Schmidt-Dannert* |
| | for lycopene synthesis in E. coli; | |
| | chloramphenicol resistant | |
| pACmod-EBI₁₄Y | Vector containing crtE, crtB, crtI₁₄, and | Present Example |
| | crtY for β-carotene synthesis in E. coli; | |
| | chloramphenicol resistant | |
| pUCmod-At-crtΔZ | A. thaliana truncated crtZ (543 bp) in | Present Example |
| | pUCmod. The 1^{st} 129 amino acids of | |
| | CrtZ were deleted. | |
| pUCmod-At-crtZ | A. thaliana crtZ (933 bp) in pUCmod; 1 | Present Example |
| | point mutation in crtZ | |
| pUCmod-At-crtZ′4 | A. thaliana crtZ (933 bp) in pUCmod; no | Present Example |
| | mutations | |
| pUCmod-At-crtZ2 | A. thaliana truncated crtZ (885 bp) in | Present Example |
| | pUCmod; The 1^{st} 16 amino acids of CrtZ | |
| | were deleted. | |
| pUCmod-At-crtZ3 | A. thaliana truncated crtZ (780 bp) in | Present Example |
| | pUCmod; The 1^{st} 51 amino acids of CrtZ | |
| | were deleted. | |
| pUCmod-Pa-crtZ | P. ananatis crtZ (528 bp) in pUCmod | Present Example |

*Schmidt-Dannert C., Umeno D., and Arnold F. H. (2000) Molecular breeding of carotenoid biosynthetic pathway. Nat. Biotechnol. 18: 750–753.

Cloning of a full-length and a truncated A. thaliana crtZ into pUCmod. To clone a full-length crtZ into pUCmod, primers At-crtZ-F1 and At-crtZ-R (Table 2) were used to amplify the gene from an A. thaliana cDNA library (Invitrogen) using 2.5 U of Taq DNA polymerase.

Primer At-crtZ-F1 contained an XbaI site followed by a Shine-Dalgarno ribosomal binding sequence (AGGAGG) and a start codon (ATG). Primer At-crtZ-R also contained an XbaI site at its 5′ end. The PCR thermal profile was (i) 3 min at 95° C., (ii) 30 cycles of 30 s at 95° C., 30 s at 55° C., and 60 s at 72° C., and (iii) 10 min at 72° C. and a hold at 4° C. The PCR product was directly cloned into the pCR2.1-TOPO vector (Invitrogen). The resultant plasmid was then digested with XbaI and released the full-length crtZ gene. The full-length crtZ gene was listed to pUCmod [Schmidt-Dannert et al.], which had previously been digested by XbaI and treated with shrimp alkaline phosphatase (SAP). The resulting plasmid was pUCmod-At-crtZ.

To clone a truncated β-carotene hydroxylase gene (crtΔZ in which nucleic acids encoding the first 129 amino acids of CrtZ were deleted) into pUCmod, primers At-crtZ-F2 and At-crtZ-R (Table 2) were used to amplify the truncated gene from the same A. thaliana cDNA library. PCR conditions were identical to those described above. Again, the PCR product was directly cloned into the pCR2.1-TOPO vector. The resultant plasmid was then digested with XbaI to release the truncated crtΔZ gene. The crtΔZ gene was ligated to pUCmod [Schmidt-Dannert et al.], which had previously been digested by XbaI and treated with SAP, forming plasmid pUCmod-At-crtΔZ.

The presence of a correctly oriented insert in both plasmids was verified by DNA sequencing using primers pUCmod-F and pUCmod-R (Table 2). A point mutation was identified in the cloned crtZ in pUCmod-At-crtZ. This mutation was corrected by using the QuikChange® Site-Directed Mutagenesis Kit (Stratagene) with primers pUC-crtZ-mutF and pUC-crtZ-mutR. The resulting plasmid was named pUCmod-At-crtZ′4.

Cloning of the Pantoea ananatis β-carotene hydroxylase (crtZ) gene. The Pantoea ananatis crtZ gene was PCR-amplified from P. ananatis genomic DNA by using Pfu DNA polymerase and primers Pa-crtZ-F plus Pa-crtZ-Co (Table 2). The PCR product was digested by XbaI and NcoI, followed by ligation to pUCmod [Schmidt-Dannert et al.], which was previously XbaI and NcoI digested and treated with SAP. The resulting plasmid was pUCmod-Pa-crtZ. The presence of an insert was verified by DNA sequencing using primers pUCmod-F and pUCmod-R (Table 2).

Construction of A. thaliana truncated β-carotene hydroxylase genes. Truncated A. thaliana β-carotene hydroxylase genes, in which either the first 16 a.a. or 51 a.a were deleted, were created as controls for comparison with the full-length A. thaliana crtZ and the P. ananatis crtZ genes. Plasmid pUCmod-At-crtZ was used as the template to amplify the truncated A. thaliana crtZ genes. All PCRs were amplified using Pfu DNA polymerase. In the first step, the truncated crtZ genes were amplified by using primer pairs At-crtZ-No2/At-crtZ-R and At-crtZ-No3/At-crtZ-R (Table 2) with the following 30-cycle thermal profile: (i) 3 min at 95° C., (ii) 30 s at 95° C., 30 s at 58° C., and 60 s at 72° C., and (iii) 10 min at 72° C. and a hold at 4° C. In the second step, 1 μL of the At-crtZ-No2/At-crtZ-R PCR product was combined with the primers At-crtZ-F4 and At-crtZ-R and the gene was re-amplified using the 30-cycle thermal profile previously mentioned. The final PCR product contained XbaI sites integrated at the 5′ and 3′ ends, which allowed cloning into pUCmod [Schmidt-Dannert et al.] creating pUCmod-At-crtZ3. Similarly, the At-crtZ-No3/At-crtZ-R PCR product generated in the first step was re-amplified using primers At-crtZ-F3 and At-crtZ-R. The final PCR product was digested with XbaI, followed by ligation to plasmid pUCmod [Schmidt-Dannert et al.] that was previously digested by XbaI and treated with SAP. The resulting plasmid was pUCmod-At-crtZ2. The presence of a correctly oriented insert in these two plasmids was verified by DNA sequencing using primers pUCmod-F and pUCmod-R (Table 2).

Batch cultivation of recombinant E. coli JM109 strains for carotenoid production. Plasmid pACmod-EBI₁₄Y was mixed with various pUCmod derivatives that contained a β-carotene hydroxylase gene. Each DNA mixture was transformed into E. coli JM109 by electroporation (settings at 1.7 kV, 200 Ω, 25 μF with a 1 mm cuvette), and transformants were selected on LB or 2×YT media containing 100 μg/mL ampicillin and 50 μg/mL chloramphenicol. A single colony from each transformation was used to inoculate 5 to 10 ml 2×YT broth containing 100 μg/mL ampicillin and 50 μg/mL chloramphenicol. The broth culture was grown overnight at 37° C. with shaking at 230 rpm. The overnight seed culture was used to inoculate 150 to 200 mL 2×YT broth containing 100 μg/mL carbenicillin and 50 μg/mL chloramphenicol (in a 500 mL baffled-flask) to a cell density of 0.01 OD₆₀₀ unit. The culture was cultivated in the dark for 48 h at 30° C. with shaking at 230 rpm.

Fermentation of recombinant E. coli JM109 strains for carotenoid production. Plasmid pACmod-EBI₁₄Y was mixed with various pUCmod derivatives that contained a β-carotene hydroxylase gene. Each DNA mixture was transformed into E. coli JM109 by electroporation, and transformants were selected on LB or 2×YT media containing 100 μg/mL ampicillin and 50 μg/mL chloramphenicol. A single colony from each transformation was used to inoculate 5 mL 2×YT broth containing 100 μg/mL ampicillin and 50 μg/mL chloramphenicol. The seed culture was grown for 12.5 h at 37° C. with shaking at 270 rpm. Ten mL of the seed culture was passaged into 200 mL 2×YT broth containing 100 μg/mL ampicillin and 50 μg/mL chloramphenicol and grown for 12.5 h at 37° C. with shaking at 270 rpm. Two 6 L New Brunswick Scientific fermentation vessels containing 3.5 L 2×YT supplemented with 100 μg/mL carbenicillin and 50 μg/mL chloramphenicol were inoculated with a 12.5 h seed culture to achieve a cell density of 0.01 OD₆₀₀. Agitation with two Rushton impellers was controlled between 200 rpm and 350 rpm to keep DO>ca. 50%. Aeration was constant at 0.51 pm with atmospheric air. Antifoam KH0402 was added as needed to control foaming. Samples of biomass were taken at regular intervals for optical density measurements and were extracted for carotenoid analysis.

Extraction and analysis of carotenoids. Fifty mL of cells were harvested by centrifugation at 10,000×g for 10 min at 4° C. The wet cells were extracted in 5 mL acetone with vigorous agitation for 2 min and the extracts were separated from the biomass by centrifugation at 10,000×g for 10 min at 4° C. The acetone extracts were kept at −80° C. for at least 1 h and a white precipitate would form. Precipitate-free acetone extracts were then collected for carotenoid analysis using a proprietary HPLC protocol.

Results and Discussion

Synthesis of β-cryptoxanthin and zeaxanthin in E. coli upon expression of A. thaliana crtZ and crtΔZ. Sun et al. previously reported that the expression of a truncated β-carotene hydroxylase (crtΔZ) gene from A. thaliana in a β-carotene-producing E. coli produced β-cryptoxanthin. In the same study, they demonstrated that expressing an A. thaliana cDNA that encoded a β-carotene hydroxylase (crtZ) gene in the same β-carotene-producing E. coli would produce zeaxanthin. We attempted to repeat their experiment by cloning A. thaliana crtZ and crtΔZ into the expression plasmid pUCmod and expressed the two genes in the β-carotene-producing E. coli JM109(pACmod-EBI₁₄Y). The expected carotenoid products for each of the recombinant strains are listed in Table 3.

TABLE 3
Expected and observed carotenoids accumulated in
recombinant E. coli strains.

| | Expected Product | |
|---|---|---|
| Plasmid | Based on Sun et al. | Observed Product |
| pACmod-EBI₁₄Y | β-carotene | β-carotene |
| pACmod-EBI₁₄Y/ | β-cryptoxanthin | zeaxanthin |
| pUCmod-At-crtΔZ | | |
| pACmod-EBI₁₄Y/ | zeaxanthin | Mostly β-cryptoxanthin, |
| pUCmod-At-crtZ | | some β-carotene |

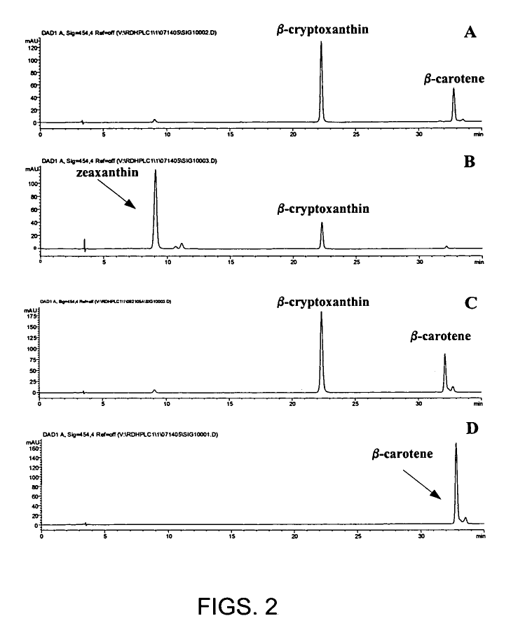

Surprisingly, HPLC analysis of JM109(pACmod-EBI₁₄Y/pUCmod-At-crtZ) cell extracts (FIG. 2A) showed that β-cryptoxanthin was the major product. Some β-carotene was also detected but little zeaxanthin was detected in the cell extracts. In contrast, the HPLC analysis of JM109(pACmod-EBI₁₄Y/pUCmod-At-crtΔZ) cell extracts (FIG. 2B) showed that most of the β-carotene was converted to zeaxanthin and β-cryptoxanthin was a minor product. Therefore, expressing the crtΔZ in a β-carotene-producing E. coli did not lead to accumulation of β-cryptoxanthin inside the cells as reported by Sun et al.

Upon inspection of the crtZ DNA sequence of pUCmod-At-crtZ, a point mutation was identified that altered amino acid 228 from phenylalanine to leucine (F228L). The point mutation was reverted to the wild-type sequence and the resulting plasmid, pUCmod-At-crtZ′4, was transformed into E. coli JM109(pACmod-EBI₁₄Y). HPLC analysis of carotenoid extracted from JM109(pACmod-EBI₁₄Y/pUCmod-At-crtZ′4) showed that the carotenoid profile (FIG. 2C) was similar to that of JM109(pACmod-EBI₁₄Y/pUCmod-At-crtZ) (FIG. 2A), with β-cryptoxanthin as the major product. Therefore, the point mutation identified in pUCmod-At-crtZ did not affect CrtZ activity and does not appear to be the cause of the difference between our data and those reported by Sun et al.

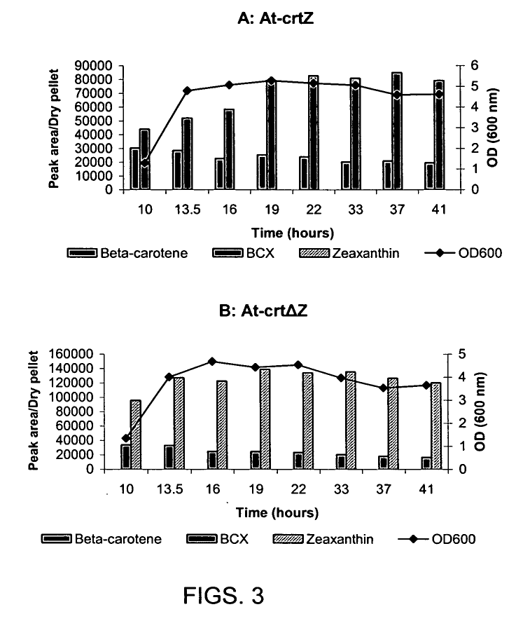

We decided to perform a batch fermentation with E. coli JM109(pACmod-EBI₁₄Y/pUCmod-At-crtZ) and JM109(pACmod-EBI₁₄Y/pUCmod-At-crtΔZ) to determine whether scale-up of the cultures would have similar results or result in complete conversion of β-cryptoxanthin to zeaxanthin. FIGS. 3A and 3B summarize cell growth and carotenoids extracted from JM109(pACmod-EBI₁₄Y/pUCmod-At-crtZ) and JM109(pACmod-EBI₁₄Y/pUCmod-At-crtΔZ) when the cells were grown in batch mode over 2 days. Cultures remained in log-phase growth up to about 20 hours. During the log-phase of growth the cells are metabolically active, but β-cryptoxanthin still accumulated inside the JM109(pACmod-EBI₁₄Y/pUCmod-At-crtZ) cells while zeaxanthin was not detected (FIG. 3A). The JM109(pACmod-EBI₁₄Y/pUCmod-At-crtΔZ) cells accumulated mostly zeaxanthin while leaving some β-cryptoxanthin (FIG. 3B) Cell densities stopped increasing at approximately 20 hours, which indicated that the cells had reached stationary phase (FIGS. 3A-3B). Around this time, the major carotenoids produced and their respective ratios remained the same for both cultures. β-cryptoxanthin was still the major product of JM109(pACmod-EBI₁₄Y/pUCmod-At-crtZ) while zeaxanthin was the major product of JM109(pACmod-EBI₁₄Y/pUCmod-At-crtΔZ). Our data therefore demonstrate that when the cells were in a metabolic active state CrtZ still converted β-carotene to β-cryptoxanthin without further transforming β-cryptoxanthin to zeaxanthin.

Carotenoid synthesis by truncated A. thaliana crtZ genes. Data indicate that the specificities of plant β-carotene hydroxylases may vary in spite of the high degree of sequence homology that exists among these protein sequences [Tian L. and DellaPenna D. (2001) Characterization of a second carotenoid β-hydroxylase gene from Arabidopsis and its relationship to the LUT1 locus. Plant Mol. Biol. 47:379-388]; [Hoshino, T., Ojima, K., and Setoguchi, Y. (2004) BHYD gene. International Publication No. WO 2004/029234 A1]; [U.S. Pat. No. 6,214,575]. Therefore, we decided to examine the primary sequence of the A. thaliana CrtZ for possible novelty.

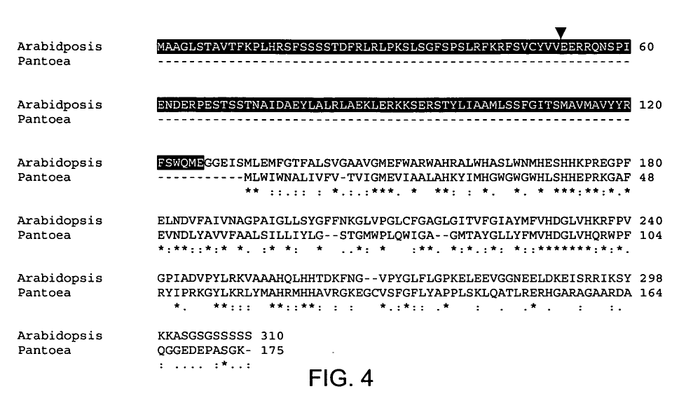

The A. thaliana β-carotene hydroxylase crtZ gene used in this application is composed of seven exons (Genbank accession no. NC_{—}003075). Exon 1 encodes the first 126 amino acids of CrtZ. Therefore, the truncated crtΔZ gene (with the first 129 amino acids deleted) reported by Sun et al. and described in this report did not contain protein sequences encoded by Exon 1 plus the first three amino acids encoded by Exon 2 of crtZ. Also, the chloroplast transit peptide prediction software ChloroP v1.1 [Center for Biological Sequence Analysis, Technical University of Denmark] predicts a chloroplast transit peptide cleavage site within Exon 1, between Val-51 and Glu-52, consistent with the presumed chloroplastic location of carotenoid biosynthetic enzymes [Cunningham F. X. Jr. and Gantt E. (1998) Genes and enzymes of carotenoid biosynthesis in plants. Annu. Rev. Plant Physiol. Plant Mol. Biol. 49:557-583]. When the A. thaliana CrtZ protein was aligned with the P. ananatis CrtZ, homologous regions were identified between the bacterial protein and the protein sequence encoded by Exons 2 to 7 of A. thaliana CrtZ (FIG. 4). The P. ananatis CrtZ, when expressed as a recombinant protein in a β-carotene-producing E. coli, is known for its capability to transform β-carotene to zeaxanthin [Misawa N., Nakagawa M., Kobayashi K., Yamano S., Izawa Y., Nakamura K., and Harashima K. (1990) Elucidation of the Erwinia uredovora carotenoid biosynthetic pathway by functional analysis of gene products expressed in Escherichia coli. J. Bacteriol. 172:6704-6712]. Since the P. ananatis CrtZ aligned well with the A. thaliana truncated CrtZ and our data repeatedly demonstrated production of zeaxanthin when this crtΔZ was expressed in a β-carotene-producing E. coli (FIGS. 2B and 3B), the conclusion of Sun et al. about the capability of crtΔZ converting β-carotene to β-cryptoxanthin appears to be incorrect. In light of this, we then hypothesized that protein sequences encoded by Exon 1 of A. thaliana crtZ might affect the CrtZ activity in E. coli and led to the production of β-cryptoxanthin from β-carotene (FIGS. 2A and 3A).

In order to test whether protein sequences encoded by Exon 1 of A. thaliana crtZ could affect CrtZ activity, we created two additional crtZ truncated genes, which were missing the first 48 and 153 nucleotides, respectively. These two truncated genes were cloned into pUCmod, forming pUCmod-crtZ2 and pUCmod-crtZ3. All of these plasmids were independently transformed into JM109(pACmod-EBI₁₄Y), and their cell extracts were analyzed for carotenoids.

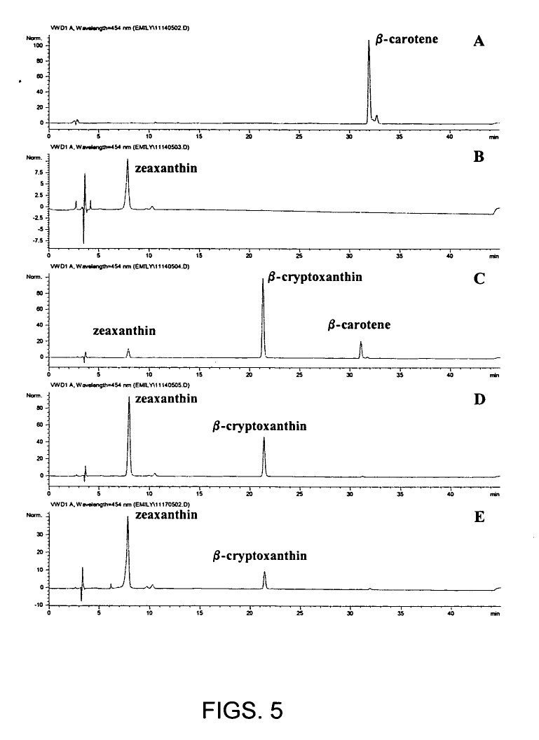

Cell extracts of the control JM109(pACmod-EBI₁₄Y/pUCmod) strain contained β-carotene (FIG. 5A). As expected, cells containing the full-length P. ananatis crtZ gene synthesized zeaxanthin (FIG. 5B), similar to the cells containing the truncated A. thaliana crtΔZ gene (FIG. 2B). Cell extracts of JM109(pACmod-EBI₁₄Y/pUCmod-At-crtZ) (FIG. 5C) showed an HPLC profile very similar to that reported previously in FIG. 2A, with β-cryptoxanthin being the major carotenoid; only small amounts of β-carotene and zeaxanthin were detected. On the other hand, cell extracts of JM109(pACmod-EBI₁₄Y/pUCmod-At-crtZ2) and JM109(pACmod-EBI₁₄Y/pUCmod-At-crtZ3) contained mostly zeaxanthin with some β-cryptoxanthin detected (FIGS. 5D and 5E). These data are intriguing because crtZ2 on pUCmod-At-crtZ2 differs from the full-length crtZ on pUCmod-At-crtZ by only 48 nucleotides. Hence, CrtZ2 lacks the first 16 amino acids of a full-length CrtZ protein from A. thaliana. However, the carotenoids produced two gene products were obviously different (FIG. 5C vs. 5D).

Summary

When a full-length A. thaliana β-carotene hydroxylase (crtZ) gene was expressed in a β-cartoene-producing E. coli, β-cryptoxanthin was found to accumulate inside the cells. Expressing a truncated version (missing the DNA encoding the first 129 amino acids) of this A. thaliana β-carotene hydroxylase (crtΔZ) gene in the same E. coli did not lead to accumulation of β-cryptoxanthin inside the cells as reported by Sun et al. Instead, zeaxanthin was the major product with little β-cryptoxanthin detected. By creating two new truncated A. thaliana crtZ gene and expressing these genes in the same β-carotene-producing E. coli, our data indicate that deletion of the first 16 amino acids of the A. thaliana CrtZ protein might have an effect on CrtZ specificity.

### EXAMPLE 2

### β-cryptoxanthin Produced in a Non-Carotenogenic Saccharomyces cerevisiae by an Engineered Metabolic Pathway Utilizing β-carotene Hydroxylase from Arabidopsis thaliana

### Materials and Methods

Materials. All reagents were of the highest purity available and were purchased from Sigma (St. Louis, Mo.), Aldrich (Milwaukee, Wis.), and Fisher Scientific (Pittsburgh, Pa.) unless otherwise noted. PCR primers were purchased from Integrated DNA Technologies, Coralville, Iowa. Pfu DNA polymerase (Stratagene, La Jolla, Calif.) and Taq DNA polymerase (Fisher Scientific, Pittsburgh, Pa.) were used in PCR reactions. Restriction endonucleases were purchased from Invitrogen (Carlsbad, Calif.), New England Biolabs (Beverly, Mass.), and Fermentas (Hanover, Mass.). T4 DNA ligase was purchased from Roche Applied Science (Indianapolis, Ind.) and Fast-Link™ DNA ligase was purchased from Epicentre (Madison, Wis.)

Bacteria, yeast, plasmids, and media. All strains and plasmids used in this study are listed in Table 4. Escherichia coli XL1-Blue (Stratagene) and E. coli JM109 were grown in 2×YT broth (per liter: Bacto tryptone, 16 g; Bacto yeast extract, 10 g; NaCl, 5 g) or low salt LB broth (per liter: Bacto tryptone, 10 g; Bacto yeast extract, 5 g; NaCl, 5 g). Ampicillin (100 μg/mL) and chloramphenicol (50 μg/mL) were added to the media for selection of various plasmid transformants in E. coli. Saccharomyces cerevisiae INVSc-1 (Invitrogen) and S. cerevisiae YPH499 (ATCC 204679) were grown in YPD media (per liter: Bacto peptone, 20 g; Bacto yeast extract, 10 g; glucose, 2 g) or a minimal medium (MM) listed in Table 5. The dropout mixture for MM was prepared by mixing 400 mg adenine, 400 mg uracil, 400 mg tryptophan, 400 mg histidine, 400 mg arginine, 400 mg methionine, 600 mg tyrosine, 1200 mg leucine, 600 mg lysine, 1000 mg phenylalanine, 4000 mg threonine, and 2000 mg aspartic acid. Minimal media formulated with dropout mixture lacking certain amino acids (Table 5) were used to select various auxotrophic yeast transformants.

TABLE 4
Bacteria, yeast, DNA, and plasmids used in this Example

| | | |
|---|---|---|
| Bacteria, Yeast, or | | |
| Plasmid | Genotype and description | Source |

Bacteria

| | | |
|---|---|---|
| Escherichia coli | Host for regular cloning; | Stratagene |
| XL1-Blue | recA1 endA1 gyrA96 thi-1 hsdR17 supE44 relA1 | |
| | lac [F′ proAB lacl^{q}ΔZM15 Tn10 (Tet^{r})] | |
| Escherichia coli | Host for expression of carotenoid biosynthetic | Kindly provided by |
| JM109 | genes; e14⁻(McrA⁻) recA1 endA1 gyrA96 thi-1 | Russell Jurenka |
| | hsdR17 (rK⁻ mK⁺) supE44 relA1 Δ(lac-proAB) [F′ | Iowa State |
| | traD36 proAB lacIqZΔM15] | University, |

Yeast

| | | |
|---|---|---|
| Saccharomyces | MAT his3D1 leu2 trp1–289 ura3–52 | Invitrogen |
| cerevisiae | | |
| INVSc-1 | | |
| Saccharomyces | MATa ura3–52 lys2–801 (amber mutation) ade2–101 | American Type |
| cerevisiae | (ochre mutation) trp1-delta63 his3-delta200 | Culture Collection, |
| YPH499 | leu2-delta1 | Manassas, VA |

Plasmids

| | | |
|---|---|---|
| pCR2.1-TOPO | Vector for direct TA-cloning of PCR product; | Invitrogen |
| | kanamycin and ampicillin resistant | |
| pUCmod-crtY | Vector containing P. ananatis crtY (1149 bp) in | Schmidt-Dannert* |
| | pUCmod; ampicillin resistant | |
| pACmod-EBI₁₄Y | Vector containing P. ananatis crtE, crtB, crtI₁₄, and | Example 1 |
| | crtY for β-carotene synthesis in E. coli; | |
| | chloramphenicol resistant | |
| pUCmod-At-crtZ | A. thaliana crtZ (933 bp) in pUCmod; ampicillin | Example 1 |
| | resistant | |
| pUCmod-Pa-crtZ | P. ananatis crtZ (528 bp) in pUCmod; ampicillin | Example 1 |
| | resistant | |
| pYES6/CT | E. coli and S. cerevisiae shuttle vector containing | Invitrogen |
| | promoter P_{GAL1} and terminator CYC1; ampicillin | |
| | and blasticidin resistant | |
| pYES6/CT-At-crtZ | A. thaliana crtZ (933 bp) in pYES6/CT; ampicillin | Present Example |
| | and blasticidin resistant | |
| pYES6/CT-Pa-crtZ | P. ananatis crtZ (528 bp) in pYES6/CT; ampicillin | Present Example |
| | and blasticidin resistant | |
| pARC145G | Vector containing P. agglomerans crtE and crtB for | U.S. Pat. |
| | β-carotene synthesis in S. cerevisiae; ampicillin | No. 5,530,188 |
| | resistant; Ura⁺ | |
| pARC145G-At-crtZ | A. thaliana crtZ (933 bp) in pARC145G for | Present Example |
| | expression in S. cerevisiae; ampicillin resistant; | |
| | Ura⁺ | |
| pARC145G-Pa-crtZ | P. ananatis crtZ (528 bp) in pARC145G for | Present Example |
| | expression in S. cerevisiae; ampicillin resistant; | |
| | Ura⁺ | |
| pARC1520 | Vector containing P. agglomerans crtI (1479 bp) | U.S. Pat. |
| | and crtY (1160 bp) for expression in S. cerevisiae; | No. 5,530,188 |
| | ampicillin resistant; Trp⁺ | |
| p423GPD | E. coli and S. cerevisiae shuttle vector containing | Mumberg‡ |
| | promoter P_{GPD} and terminator CYC1; ampicillin | |
| | resistant; His⁺ | |
| p423GPD-crtY | P. ananatis crtY (1149 bp) in p423GPD for | Present Example |
| | expression in S. cerevisiae; ampicillin resistant; | |
| | His⁺ | |
| pESC-URA | E. coli and S. cerevisiae shuttle vector containing | Stratagene |
| | promoter P_{GAL1} and terminator CYC1; ampicillin | |
| | resistant; Ura⁺ | |
| pESC-URA-crtI | P. agglomerans crtI (1479 bp) in pESC-URA for | Present Example |
| | expression in S. cerevisiae; ampicillin resistant; | |
| | Ura⁺ | |

*Schmidt-Dannert C., Umeno D., and Arnold F. H. (2000) Molecular breeding of carotenoid biosynthetic pathway. Nat. Biotechnol. 18: 750–753
‡Mumberg D., Muller R., and Funk M. (1995) Yeast vectors for the controlled expression of heterologous proteins in different genetic backgrounds. Gene 156: 119–122

TABLE 5
Minimal Media (MM) recipe, 1 liter

| Constituent | Amount (mg) |
|---|---|
| Yeast nitrogen base without amino acids | 6700 |
| Glucose or galactose | 2000 |
| Dropout mixture* | 720 |

*Minimal medium 2: without uracil and tryptophan; Minimal medium 3: without uracil, tryptophan, and histidine

Cloning of an Arabidopsis thaliana β-carotene hydroxylase (At-crtZ) gene into pARC145G. To clone the full-length A. thaliana crtZ gene into pARC145G, primers YES-AtZ-F and YES-AtZ-R (Table 6) were used to amplify the gene from pUCmod-At-crtZ using 2.5 U of Pfu Turbo DNA polymerase. Primer YES-AtZ-F contained a HindIII site and primer YES-AtZ-R contained an XhoI site at their 5′ ends. The PCR thermal profile was (i) 3 min at 95° C., (ii) 30 cycles of 30 s at 95° C., 30 s at 55° C., and 60 s at 68° C., (iii) 10 min at 72° C. and a hold at 4° C. The PCR product was digested with HindIII and XhoI, cleaned using a QIAquick PCR Purification kit (Qiagen), and ligated to pYES6/CT, which had been previously digested by HindIII and XhoI, using Fast-Link™ DNA ligase. The resultant plasmid was pYES6/CT-At-crtZ. DNA sequencing of pYES6/CT-At-crtZ using primers pYES6-F and pYES6-R (Table 6) verified the presence of a correctly oriented insert in the plasmid. Primers Pgal-cass-F and Pgal-cass-R (Table 6), which contained an SphI recognition sequence at their 5′ ends, were then used to amplify the P_{GAL1}-A. thaliana crtZ-CYC1 transcription terminator cassette from pYES6/CT-At-crtZ using a thermal profile of (i) 3 min at 95° C., (ii) 30 cycles of 30 s at 95° C., 30 s at 58° C. and 90 s at 72° C., (iii) 10 min at 72° C. and a hold at 4° C. The PCR product was digested with SphI, purified using a QIAquick PCR Purification kit, and ligated to SphI-digested pARC145G. The resultant plasmid was pARC145G-At-crtZ.

Cloning of the Pantoea ananatis crtZ gene into pARC145G. The P. ananatis crtZ gene was PCR-amplified from pUCmod-Pa-crtZ by using Pfu Turbo DNA polymerase and primers YES-PaZ-F and YES-PaZ-R (Table 6). Primer YES-PaZ-F contained a HindIII site and primer YES-PaZ-R contained an XhoI site at their 5′ ends. The PCR thermal profile was (i) 3 min at 95° C., (ii) 30 cycles of 30 s at 95° C., 30 s at 55° C., and 60 s at 68° C., (iii) 10 min at 72° C. and a hold at 4° C. The PCR product was digested with HindIII and XhoI, purified using a QIAquick PCR Purification kit (Qiagen), and ligated to pYES6/CT, which had been previously digested by HindIII and XhoI. The resultant plasmid was pYES6/CT-Pa-crtZ. DNA sequencing using primers pYES6-F and pYES6-R (Table 6) verified the presence of a correctly oriented insert in the plasmid. After that, primers Pgal-cass-F and Pgal-cass-R (Table 6), which had SphI overhangs at their 5′ ends, were used to amplify the P_{GAL1}-P. ananatis crtZ-CYC1 transcription terminator cassette from pYES6/CT-Pa-crtZ. The PCR product was digested with SphI, purified using a QIAquick PCR Purification kit, and ligated to SphI-digested pARC145G. The resultant plasmid was pARC145G-Pa-crtZ.

Cloning of the P. ananatis lycopene cyclase (crtY) gene into p423GPD. The lycopene β-cyclase (crtY) gene from P. ananatis was amplified from pUCmod-crtY using Pfu Turbo DNA polymerase and primers crtY-F and crtY-R (Table 6). Primer crtY-F contained an EcoRI site at its 5′ end and primer crtY-R contained a SalI site at its 5′ end. The PCR thermal profile was (i) 3 min at 95° C., (ii) 30 cycles of 30 s at 95° C., 30 s of 55° C., and 1 min 15 s at 68° C., (iii) 10 min at 72° C. and a hold at 4° C. After completion of the PCR reaction, 2.5 U Taq DNA polymerase was added to the PCR product and the mixture was incubated for 10 min at 72° C. This additional incubation in the presence of Taq DNA polymerase ensured the addition of a single dATP to the 3′-ends of the PCR of product and allowed direct cloning of the PCR product into pCR2.1-TOPO (Invitrogen). DNA Sequencing verified successful cloning of crtY into pCR2.1-TOPO. After that, crtY was digested from the pCR2.1-TOPO vector with EcoRI and SalI, purified using a QIAquick Gel Purification kit, and ligated to p423GPD, which had been previously digested by EcoRI and SalI. The resulting plasmid was p423GPD-crtY. The presence of the correct insert was confirmed by sequencing the plasmid using internal primers F1-crtY, R1-crtY, F2-crtY, and R2-crtY (Table 6).

Cloning of the P. agglomerans phytoene dehydrogenase (crtI) gene into pESC-URA. The Pantoea agglomerans phytoene dehydrogenase (crtI) gene was released from pARC1520 by SalI and was gel-purified. The purified insert was then amplified using Pfu Turbo DNA polymerase and primers crtI-F and crtI-R (Table 6). Both primers contained a SalI site at the 5′ ends and crtI-F also contained a yeast “Kozak” sequence [Hamilton et al.] preceding the start codon for crtI. The PCR thermal profile was (i) 3 min at 95° C., (ii) 30 cycles of 30 s at 95° C., 30 s of 55° C., and 1 min 15 s at 68° C., (iii) 10 min at 72° C. and a hold at 4° C. The PCR product was incubated in the presence of 2.5 U Taq DNA polymerase for 10 min at 72° C., which allowed direct cloning of the PCR product into pCR2.1-TOPO by using a TOPO TA Cloning® kit. DNA Sequencing verified the presence of insert in the plasmid. The crtI gene was released from the pCR2.1-TOPO vector by SalI, gel-purified, and ligated to pESC-URA that was previously digested by SalI digested. The resulting plasmid was pESC-URA-crtI. DNA sequencing using internal primers F1-crtI, R1-crtI, F2-crtI, and R2-crtI (Table 6) verified the presence of a complete and correctly oriented insert in the plasmid.

Batch cultivation of recombinant S. cerevisiae strains for carotenoid production. Various combinations of carotenogenic plasmids (Table 7) were transformed into S. cerevisiae INVSc-1 or YPH499 using the S.c. EasyComp™ Transformation kit (Invitrogen). Transformants were selected on MM agar containing 2% glucose and the appropriate dropout mixture (Table 5). A single transformant colony was then used to inoculate 10 mL of MM with 2% glucose and appropriate dropout mixture. The culture was grown 24 to 48 h at 30° C. with shaking at 230 rpm. The seed culture was used to inoculate 120 to 125 mL MM broth containing 2% galactose and the appropriate dropout mixture (in a 500 mL baffled-flask) to a cell density of 0.04 OD₆₀₀ units. The culture was cultivated in the dark for 72 to 96 h at 30° C. with shaking at 230 rpm.

TABLE 7
Plasmid combinations expressed in S. cerevisiae in this Example

| | | Media |
|---|---|---|
| Host and Plasmid Combinations | Expressed Genes¹ | Used² |
| S. cerevisiae INVSc-1 | | |
| pARC1520 + pARC145G | crtE, crtI, crtB, crtY | MM#2 |
| pARC1520 + pARC145G-At-crtZ | crtE, crtI, crtB, crtY, At-crtZ | MM#2 |
| pARC1520 + pARC145G-Pa-crtZ | crtE, crtI, crtB, crtY, Pa-crtZ | MM#2 |
| S. cerevisiae YPH499 | | |
| pARC1520 + pARC145G | crtE, crtI, crtB, crtY | MM#2 |
| pARC1520 + pARC145G-At-crtZ | crtE, crtI, crtB, crtY, At-crtZ | MM#2 |
| pARC1520 + pARC145G-Pa-crtZ | crtE, crtI, crtB, crtY, Pa-crtZ | MM#2 |
| pARC145G + pESC-URA-crtI | crtE, crtI, crtB | MM#2 |
| pARC145G + pESC-URA-crtI + p423GPD-crtY | crtE, crtI, crtB, crtY | MM#3 |
| pARC145G-At-crtZ + pESC-URA-crtI + p423GPD- | crtE, crtI, crtB, crtY, At-crtZ | MM#3 |
| crtY | | |
| pARC145G-Pa-crtZ + pESC-URA-crtI + p423GPD- | crtE, crtI, crB, crtY, Pa-crtZ | MM#3 |
| crtY | | |
| pARC1520 + pARC145G + p423GPD-crtY | crtE, crtI, crtB, crtY, Pa-crtY | MM#3 |
| pARC1520 + pARC145G-At-crtZ + p423GPD- | crtE, crtI, crtB, crtY, Pa-crtY, | MM#3 |
| crtY | At-crtZ | |
| pARC1520 + pARC145G-Pa-crtZ + p423GPD- | crtE, crtI, crtB, crtY, Pa-crtY, | MM#3 |
| crtY | Pa-crtZ | |

¹All crt genes are from P. agglomerans except, At-crtZ (from A. thaliana), Pa-crtZ (from P. ananatis), and Pa-crtY (from P. ananatis).
²Media recipes are detailed in Table 5.

Extraction and analysis of carotenoids. Fifty mL of cells were harvested by centrifugation at 10,000×g for 10 min at 4° C. The cell pellets were frozen at −80° C. and lyophilized. The dry cell pellets were vigorously agitated for 5 min in 10 mL acetone with 20 g of 400-600 μm glass beads (Fisher). The extracts were separated from the biomass and beads by centrifugation at 20,000×g for 10 min at 4° C. The acetone extracts were finally filtered through a 0.45-μm nylon membrane to remove any cellular debris. Filtered acetone extracts were then analyzed for carotenoids using a proprietary HPLC protocol.

Results and Discussion

Example 1 describes the production of β-cryptoxanthin in a carotenogenic E. coli expressing a β-carotene hydroxylase (crtZ) gene from A. thaliana. The limited conversion of β-cryptoxanthin to zeaxanthin by A. thaliana CrtZ was a novel and desirable property. In order to determine if this property of A. thaliana crtZ is a universal function that applies to other host organisms, it was decided to attempt to express this gene in a β-carotene-producing S. cerevisiae. Many yeast strains are considered GRAS organisms, which is desirable for the production of carotenoids for the dietary supplement market.

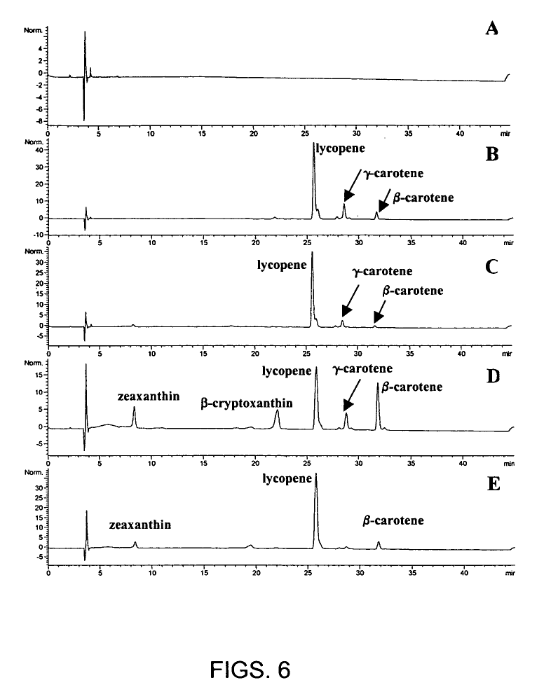

Ausich et. al. [U.S. Pat. No. 5,530,188] previously engineered a β-carotene-producing yeast strain YPH499(pARC1520/pARC145G). The two plasmids contained carotenoid synthetic genes isolated from Pantoea agglomerans. As disclosed in Example I, we cloned the full-length A. thaliana crtZ and the P. ananatis crtZ, which respectively produced β-cryptoxanthin and zeaxanthin from β-carotene in E. coli into pARC145G. The pARC145G derivatives, pARC145G-AtZ and pARC145G-PaZ, were transformed into INVSc-1(pARC1520). Surprisingly, HPLC analyses showed that INVSc-1 (pARC1520/pARC145G) cell extracts contained no detectable carotenoids (FIG. 6A). Meanwhile, HPLC analyses of INVSc-1(pARC1520/pARC145G-At-crtZ) and INVSc-1(pARC1520/pARC145G-Pa-crtZ) cell extracts (FIGS. 6B and 6C) showed lycopene as the major product with some γ-carotene and β-carotene as minor products. Neither β-cryptoxanthin nor zeaxanthin were detected. Since Ausich et al. used YPH499 instead of INVSc-1 as the host for expressing carotenogenic genes, we repeated the experiment in YPH499 to test whether the lack of conversion of lycopene was host-specific. As in the INVSc-1 background, no carotenoid could be detected in YPH499(pARC1520/pARC145G). Meanwhile, a small amount of β-cryptoxanthin (peak area 138) and zeaxanthin (peak area 87) were detected in YPH499(pARC1520/pARC145G-AtZ) (FIG. 6D). A small zeaxanthin peak, but no β-cryptoxanthin, could be detected in YPH499(pARC1520/pARC145G-PaZ) (FIG. 6E). The production of more β-cryptoxanthin than zeaxanthin in YPH499(pARC1520/pARC145G-AtZ) (FIG. 6D) agreed with the results in a recombinant E. coli that expressed the At-crtZ gene as disclosed in Example I.

However, some of the results were unexpected. First, it is surprising that carotenoids were not detected in the control strains (FIG. 6A), while lycopene, γ-carotene, and β-carotene were detected in strains in which a crtZ gene was present. A barrier seems to exist which blocks the production of lycopene from phytoene in the control strains. When CrtZ is present inside the cells, CrtZ seems to “pull” the reaction over this barrier and results in formation of lycopene (FIGS. 6B-6E). Second, we are surprised by the inefficient conversion of lycopene to γ- and β-carotene in the tested strains that contained crtZ genes. As β-carotene was the substrate for β-cryptoxanthin and zeaxanthin, a lack of β-carotene production would limit the yield of β-cryptoxanthin or zeaxanthin. The build-up of lycopene inside the cells suggested low lycopene β-cyclase (CrtY) activity in vivo. Inefficient expression of the P. agglomerans crtY in yeast might be one possibility. Alternatively, over expression of too many heterologous genes inside the same host organism could be problematic. The recombinant yeast strains were expressing 5 different crt genes and all of them were regulated by the inducible P_{GAL1} and P_{GAL10} promoters. The P_{GAL} promoters are strong promoters that are commonly used for producing large quantities of proteins in yeast. Over-production of these recombinant proteins, however, might become a metabolic burden to the cells and have a negative impact on the cells for carotenoid synthesis.

To address these possibilities, we decided to construct 2 new plasmids. Plasmid p423GPD-crtY contained a P. ananatis crtY, regulated by a weaker constitutive glyceraldehyde 3-phosphate dehydrogenase promoter. P. ananatis crtY is a lycopene β-cyclase gene commonly used in carotenogenic research. Yamano et al. [Yamano S., Ishii T., Nakagawa M., Ikenaga H. and Misawa N. (1994) Metabolic engineering for production of β-carotene and lycopene in Saccharomyces cerevisiae. Biosci. Biotech. Biochem. 58:1112-1114] previously constructed a yeast strain containing crt genes from P. ananatis and reported efficient conversion of lycopene to β-carotene with very limited build-up of intermediates. A second plasmid, pESC-URA-crtI, contained the P. agglomerans crtI sub-cloned from pARC1520 and the gene was still regulated by P_{GAL1}. Plasmids pESC-URA-crtI, p423GPD-crtY, and pARC145G derivatives were transformed into strain YPH499. The resultant strains should have a weaker metabolic load than the original YPH499(pARC145G/pARC1520), since crtY expression was controlled by a much weaker promoter. In a separate experiment, plasmid p423GPD-crtY was transformed into YPH499 together with pARC1520 and pARC145G derivatives. The resultant strains would have additional copies of crtY, which might address possible insufficient expression of crtY. The expressed plasmid combinations and their expected products are summarized in Table 9.

TABLE 9
Expected and observed carotenoids accumulated in
recombinant S. cerevisiae YPH499

| Plasmid Combination | Expected Product | Observed Product |
|---|---|---|
| pARC145G-At-crtZ + | β-cryptoxanthin | β-cryptoxanthin, |
| pESC-URA-crtI + p423GPD- | | zeaxanthin |
| crtY | | |
| pARC145G-Pa-crtZ + | zeaxanthin | zeaxanthin |
| pESC-URA-crtI + p423GPD- | | |
| crtY | | |
| pARC145G-At-crtZ + | β-cryptoxanthin | β-cryptoxanthin, |
| pARC1520 + | | zeaxanthin |
| p423GPD-crtY | | |
| pARC145G-Pa-crtZ + | zeaxanthin | zeaxanthin |
| pARC1520 + | | |
| p423GPD-crtY | | |

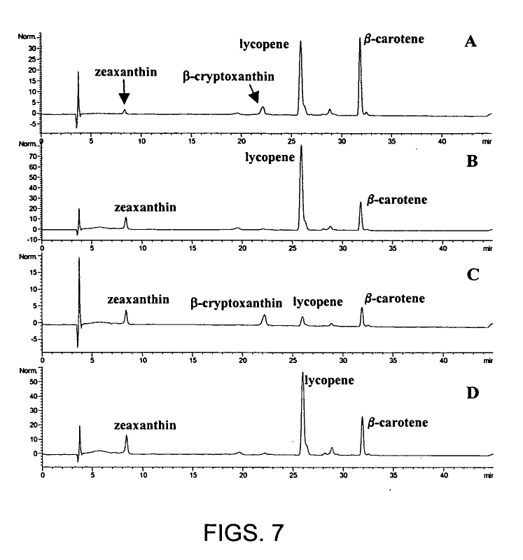

HPLC analyses of YPH499(pARC145G-At-crtZ/pESC-URA-crtI/p423GPD-crtY) (FIG. 7A) cell extracts showed lycopene and β-carotene as major products along with some β-cryptoxanthin and zeaxanthin. The peak area ratio of β-cryptoxanthin to zeaxanthin was about 3.8:1.0. Meanwhile, YPH499(pARC145G-Pa-crtZ/pESC-URA-crtI/p423GPD-crtY) cell extracts contained lycopene, β-carotene, and zeaxanthin. There was no measurable amount of β-cryptoxanthin produced (FIG. 7B). YPH499(pARC1520/pARC145G-At-crtZ/p423GPD-crtY) (FIG. 7C) cell extracts showed a low level production of carotenoids overall, but slightly more β-cryptoxanthin was produced than zeaxanthin with a peak area ratio of 1.3:1.0. Finally, cell extracts from YPH499(pARC1520/pARC145G-Pa-crtZ/p423GPD-crtY) (FIG. 7D) contained lycopene as the major product with β-carotene and zeaxanthin being the minor products. Again this P. ananatis crtZ-containing strain did not produce β-cryptoxanthin.

The results from this experiment agreed with those in E. coli; more β-cryptoxanthin than zeaxanthin was produced in cells containing the full-length A. thaliana crtZ, while cells containing the P. ananatis crtZ produced only zeaxanthin.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art who have the disclosure before them will be able to make modifications and variations therein without departing from the scope of the invention.

We claim:

1. A method for preparing β-cryptoxanthin, comprising culturing a carotenogenic microorganism transformed with the β-carotene hydroxylase gene from Arabidopsis thaliana in a medium and recovering β-cryptoxanthin from the resultant culture.

2. The method of claim 1, wherein the microorganism is selected from the group consisting of bacteria and yeasts.

3. A method as defined in claim 2, wherein the bacteria are E. coli.

4. A method as defined in claim 2, wherein the yeasts are S. cerevisiae.

5. A method of producing β-cryptoxanthin as the dominant carotenoid of a carotenogenic microorganism, comprising transforming the microorganism with the β-carotene hydroxylase gene from Arabidopsis thaliana and culturing the transformed microorganism in a medium.

6. The method of claim 5, wherein the microorganism is selected from the group consisting of bacteria and yeasts.

7. A method as defined in claim 6, wherein the bacteria are E. coli.

8. A method as defined in claim 6, wherein the yeasts are S. cerevisiae.

9. A method for preparing zeaxanthin, comprising culturing a carotenogenic microorganism transformed with the truncated β-carotene hydroxylase gene from Arabidopsis thaliana in a medium and recovering zeaxanthin from the resultant culture.

10. The method of claim 9, wherein the microorganism is selected from the group consisting of bacteria and yeasts.

11. A method as defined in claim 10, wherein the bacteria are E. coli.

12. A method as defined in claim 10, wherein the yeasts are S. cerevisiae.

13. A method of producing zeaxanthin as the dominant carotenoid of a carotenogenic microorganism, comprising transforming the microorganism with the truncated β-carotene hydroxylase gene from Arabidopsis thaliana and culturing the transformed microorganism in a medium.

14. The method of claim 13, wherein the microorganism is selected from the group consisting of bacteria and yeasts.

15. A method as defined in claim 14, wherein the bacteria are E. coli.

16. A method as defined in claim 14, wherein the yeasts are S. cervisiae.
